(19) United States
(12) Patent Application Publication
(10) Pub. No.: US 2009/0069292 A1
(43) Pub. Date: Mar. 12, 2009

(54) Liquid Formulations

(75) Inventors: Daniel Korey, Wilmington, DE (US); Karen B. Main, Wilmington, DE (US); Richard J.R. Creekmore, Wilmington, DE (US); Jean M. Surian, Wilmington, DE (US); Vivian Y. Bi, Wilmington, DE (US)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/093,957

(22) PCT Filed: Nov. 18, 2006
(86) PCT No.: PCT/US2006/061072

§ 371 Date: Nov. 7, 2008

Related U.S. Application Data
(60) Provisional application No. 60/737,885, filed on Nov. 18, 2005.

(51) Int. Cl. A61K 31/554; A61P 25/18

(52) U.S. Cl. 514/211.13

(57) ABSTRACT

The present invention is directed to liquid formulations of the pharmaceutical compound 1 1-piperazin-1-yldibenzo[b,f][1,4]thiazepine as well as preparations, and pharmaceutical uses thereto.

### FIELD OF THE INVENTION

The present invention is directed to liquid formulations containing the pharmaceutical compound 11-piperazin-1-yldibenzo[b,f][1,4]thiazepine, as well as pharmaceutical uses thereof.

### BACKGROUND OF THE INVENTION

A goal of antipsychotic drug development has been to develop agents with increased efficacy and safety along with fewer of the side effects commonly associated with the older antipsychotic medications. Quetiapine fumarate is described in U.S. Pat. No. 4,879,288, which is incorporated herein by reference. Quetiapine fumarate is able to treat both the positive (hallucinations, delusions) and negative symptoms (emotional withdrawal, apathy) of psychosis and is associated with fewer neurological and endocrine related side effects compared to older agents. Quetiapine fumarate has also been associated with a reduction in hostility and aggression. Quetiapine fumarate is associated with fewer side effects such as EPS, acute dystonia, acute dyskinesia, as well as tardive dyskinesia. Quetiapine fumarate has also helped to, enhance patient compliance with treatment, ability to function and overall quality of life, while reducing recidivism. P. Weiden et al., Atypical antipsychotic drugs and long-term outcome in schizophrenia, 11 J. Clin. Psychiatry, 53-60, 57 (1996). Because of quetiapine fumarate's enhanced tolerability profile its use is particularly advantageous in the treatment of patients that are hypersensitive to the adverse effects of antipsychotics (such as elderly patients).

Derivatives of 11-(piperazin-1-yl)dibenzo[b,f][1,4]-thiazepines and related compounds including metabolites of quetiapine were prepared and evaluated in E. Warawa et al. Behavioral approach to nondyskinetic dopamine antagonists: identification of Seroquel, 44, J. Med. Chem., 372-389 (2001). Quetiapine metabolism has been reported in C. L. Devane et al. Clin. Pharmacokinet., 40(7), 509-522 (2001) wherein the structure of 11-piperazin-1-yldibenzo[b,f][1,4]thiazepine (see Formula I below) was shown in FIG. 1. This compound was reported by Schmutz et al. in U.S. Pat. No. 3,539,573. This compound has also been used in processes for preparing quetiapine as reported in U.S. Pat. No. 4,879,288. It has now been found that 11-piperazin-1-yldibenzo[b,f][1,4]thiazepine is a circulating metabolite of quetiapine in humans.

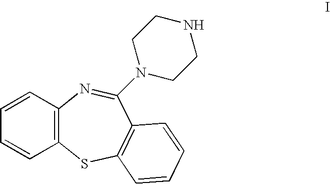

### SUMMARY OF THE INVENTION

The present invention provides liquid formulations comprising 11-piperazin-1-yldibenzo[b,f][1,4]thiazepine in a liquid vehicle and optionally further comprising at least one pharmaceutical excipient selected from a buffer, an antioxidant, a chelating agent, a preservative, a tonicity adjuster, a cyclodextrin, a surfactant, a suspending agent, a wetting agent, a stabilizer, a flocculating agent, a sweetener, a flavoring, a colorant, and a cosolvent.

The present invention further provides methods of treating at least one symptom or condition associated with but not limited to: 1) Schizophrenia and other Psychotic Disorders including but not limited to Psychotic Disorder, Schizophreniform Disorder, Schizoaffective Disorder, Delusional Disorder, Brief Psychotic Disorder, Shared Psychotic Disorder, and Psychotic Disorder Due to a General Medical Condition; 2) Dementia and other Cognitive Disorders; 3) Anxiety Disorders including but not limited to Panic Disorder Without Agoraphobia, Panic Disorder With Agoraphobia, Agoraphobia Without History of Panic Disorder, Specific Phobia, Social Phobia, Obsessive-Compulsive Disorder, Postraumatic Stress Disorder, Acute Stress Disorder, Generalized Anxiety Disorder and Generalized Anxiety Disorder Due to a General Medical Condition; 4) Mood Disorders including but not limited to a) Depressive Disorders, including but not limited to Major Depressive Disorder and Dysthymic Disorder and b) Bipolar Depression and/or Bipolar mania including but not limited to Bipolar I Disorder, including but not limited to those with manic, depressive or mixed episodes, and Bipolar II Disorder, c) Cyclothymic Disorder, d) Mood Disorder Due to a General Medical Condition; 5) Sleep Disorders; 6) Disorders Usually First Diagnosed in Infancy, Childhood, or Adolescence including but not limited to Mental Retardation, Learning Disorders, Motor Skills Disorder, Communication Disorders, Pervasive Developmental Disorders, Attention-Deficit and Disruptive Behavior Disorders, Feeding and Eating Disorders of Infancy or Early Childhood, Tic Disorders, and Elimination Disorders; 7) Substance-Related Disorders including but not limited to Substance Dependence, Substance Abuse, Substance Intoxication, Substance Withdrawal, Alcohol-Related Disorders, Amphetamine (or Amphetamine-Like)-Related Disorders, Caffeine-Related Disorders, Cannabis-Related Disorders, Cocaine-Related Disorders, Hallucinogen-Related Disorders, Inhalant-Related Disorders, Nicotine-Related Disorders, Opioid-Related Disorders, Phencyclidine (or Phencyclidine-Like)-Related Disorders, and Sedative-, Hypnotic- or Anxiolytic-Related Disorders; 8) Attention-Deficit and Disruptive Behavior Disorders; 9) Eating Disorders; 10) Personality Disorders including but not limited to Obsessive-Compulsive Personality Disorder; and 11) Impulse-Control Disorders, comprising administering to a mammal a therapeutically effective amount of a formulation of the invention.

### BRIEF DESCRIPTION OF THE DRAWINGS

FIG. 1 depicts an XRPD pattern consistent with Form A.

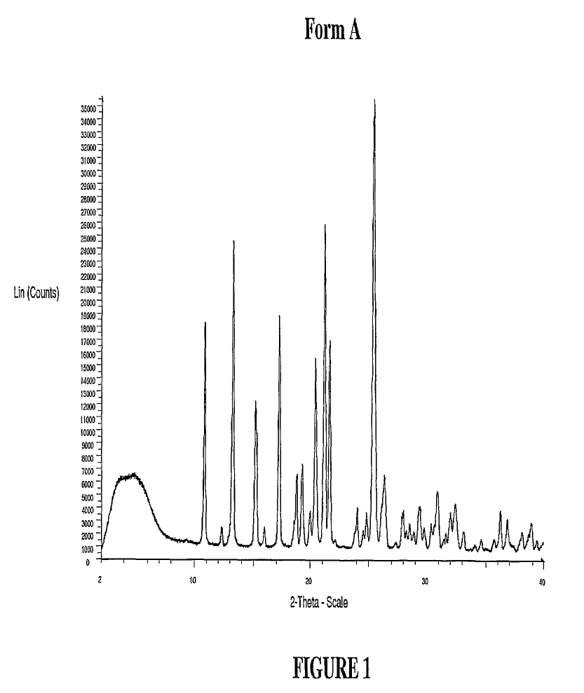

FIG. 2 depicts TGA and DSC data consistent with Form A.

FIG. 3 depicts DVS data consistent with Form A.

### DETAILED DESCRIPTION

The compound of Formula I is a dibenzothiazepine that has shown antidopaminergic activity. It has been shown to interact with a broad range of neurotransmitter receptors but has a higher affinity for serotonin (5-HT₂) receptors relative to dopamine (D₂) receptors in the brain. Preliminary positron emission topography (PET) scans of primate subjects showed that the compound of Formula I reaches the brain and occupies D1, D₂, 5-HT_{2A}, and 5-HT_{1A} receptors and the 5HT Transporter. However, the compound of Formula I was not shown to be efficacious in a mouse standard apomorphine swim test (p.o.) and in a rat D-Amphetamine locomotor activity test (s.c.).

The compound of Formula I has also been shown to have partial 5HT_{1A} agonist activity and has shown in-vivo efficacy in mouse and rat models for depression. The compound of Formula I may be used as an antipsychotic with a reduction in the potential to cause side effects such as acute dystonia, acute dyskinesia, as well as tardive dyskinesia typically seen with antipsychotics. Results generated from alpha receptor binding data further suggest that the compound of Formula I will have improved tolerability over that of quetiapine and suggest that one would observe a reduced incidence of hypotension. Further the compound of Formula I may be used to treat patients of all ages and is advantageous in the treatment of elderly patients.

The present invention provides liquid formulations containing 11-piperazin-1-yldibenzo[b,f][1,4]thiazepine in a liquid vehicle. Liquid formulations can be in the form of solutions or suspensions. Liquid formulations can be suitable for oral administration or injection. Additional ingredients of the liquid formulations of the invention can optionally include, for example, at least one pharmaceutical excipient selected from a buffer, an antioxidant, a chelating agent, a preservative, a tonicity adjuster, a cyclodextrin, a surfactant, a suspending agent, a wetting agent, a stabilizer, a flocculating agent, a sweetener, a flavoring, a colorant, a cosolvent, and other ingredients. Oral liquid formulations can contain taste-masking ingredients such as sweeteners (artificial and/or natural) and flavorings.

The liquid vehicle can be any pharmaceutically acceptable liquid carrier including water, oils, (e.g., edible oils), and mixtures thereof. Example oils include sesame oil, soybean oil, other plant-derived oils and mixtures thereof.

The liquid formulations can contain any suitable amount of the active ingredient 11-piperazin-1-yldibenzo[b,f][1,4]thiazepine. In some embodiments, 11-piperazin-1-yldibenzo[b,f][1,4]thiazepine is present in an amount of about 0.005 to about 60, about 0.005 to about 50, about 0.005 to about 10, about 0.005 to about 5, or about 0.005 to about 2% w/v. In some embodiments, 11-piperazin-1-yldibenzo[b,f][1,4]thiazepine is present in an amount of about 0.005, about 0.01, about 0.05, about 0.1, about 0.9, about 1.0, about 1.4, about 1.5, about 5, or about 6% w/v.

A buffer can be optionally added to the liquid, for example, to maintain pH in a desired range or enhance the solubility of the pharmaceutically active agent. Suitable buffers are those that are not chemically reactive with other ingredients and are present in amounts sufficient to provide the desired degree of pH buffering. In some embodiments, the buffer is selected to assist in maintaining an acidic pH of the liquid formulation. For example, the pH can be from about 3 to about 6. In some embodiments, the pH is about 4, about 4.5, or about 5. Example pharmaceutically acceptable buffers suitable for maintaining an acidic pH include lactic acid, citric acid, acetic acid, ascorbic acid, and the like. These acids can be titrated with any appropriate base (e.g., sodium hydroxide, sodium acetate, disodium phosphate, and the like) to reach the desired pH. Other buffers include potassium phosphate and sodium phosphate. The amount of buffer can range from about 0 to about 5, about 0 to about 3, about 0.1 to about 3, about 0.1 to about 1, or about 1 to about 3% w/v. In some embodiments, the buffered vehicle can be about 0.1 to about 0.5, about 0.1 to about 0.3, or about 0.3 M.

Suitable antioxidants are known in the art and include, for example, citric acid, tocopherols, and the like. The amount of antioxidant can be present, for example, in an amount of about 0 to abut 3, 0 to about 1, or 0.1 to about 1% w/v.

Suitable chelating agents include any pharmaceutically acceptable sequestering agent such as EDTA (ethylenediaminetetraacetic acid) in an amount of about 0 to about 0.1% w/v.

Preservatives include but are not limited to sodium benzoate, potassium sorbate, salts of edetate (also known as salts of ethylenediaminetetraacetic acid, or EDTA, such as disodium edetate), parabens (such as methyl, ethyl, propyl and butyl p-hydroxybenzoic acids esters or mixtures thereof) or mixtures thereof. Sodium benzoate, propylparaben, butylparaben or mixtures thereof are preferred preservative ingredients and may be added to a liquid formulation although other pharmaceutically acceptable preservatives can be substituted. Preservatives can be present in amounts of up to about 1 gram per 100 mL. Preferably, an individual preservative may be present in an amount in the range of from about 0.015 to about 0.5 gram per 100 mL. Preferably, a preservative such as propylparaben, butylparaben or mixtures thereof is present in a range of from about 0.01 to about 0.05 gram per 100 mL. More preferably, about 0.006 gram per 100 mL of a preservative selected from propylparaben, butylparaben or mixtures thereof is present. A preservative such as sodium benzoate can be optionally present in a range of from about 0.1 to about 0.5 gram per 100 mL. More preferably, about 0.2 gram per 100 mL sodium benzoate is present. In some embodiments, the liquid formulation comprises about 0 to about 5% w/v preservative.

Tonicity adjuster can be added to the liquid formulations of the invention to achieve a desired tonicity. In some embodiments, the desired tonicity is substantially the same as for blood plasma, such as for formulations suitable for injection. Accordingly, suitable tonicity can be about 300 mOsm/kg. Example tonicity adjusters include salts (e.g., sodium chloride), dextrose, and the like. For example, the formulations of the invention can contain about 0 to about 10, about 0 to about 6, about 0.5 to about 6, or about 0.9 to about 5.5% w/v of tonicity adjuster.

In some embodiments, the formulations of the invention include a cyclodextrin such as, for example, hydroxypropyl-β-cyclodextrin or the like in an amount of about 0 to about 70, about 1 to about 65, or about 1 to about 45% w/v.

Surfactants can be included in the formulations of the invention. Suitable surfactants include, for example, sorbitan oleate ester, polyoxyethylene sorbitan monooleate, and the like. The surfactant can be present in an amount of about 0 to about 10, about 0 to about 5, or about 0.1 to about 5% w/v.

Cosolvents include other liquids in addition to the liquid vehicle that contribute to the liquid volume of the formulation. Example cosolvents include organic solvents including alcohols such as ethanol and other pharmaceutically acceptable liquids. In some embodiments, the liquid formulation contains about 0 to about 75, about 0 to about 50, about 0 to about 30, about 0 to about 20, or about 0 to about 10% w/v cosolvent.

Artificial sweeteners that may be used include, and are not limited to, aspartame, acesulfame potassium, saccharin, saccharin sodium, sucralose or mixtures thereof. The taste masking effective amount of aspartame has a range of from about 0.15 to about 8 grams per 100 mL, The taste masking effective amount of acesulfame potassium has a range of from about 0.15 to about 8 grams per 100 mL. The taste masking effective amount of saccharin has a range of from about 0.08 to about 3 grams per 100 mL. The taste masking effective amount of saccharin sodium has a range of from about 0.1 to about 5 grams per 100 mL.

Flavoring agents that may be used include, and are not limited to, natural flavors, natural fruit flavors, artificial flavors, artificial fruit flavors, flavor enhancers or mixtures thereof. Natural flavors, artificial flavors or mixtures thereof include, and are not limited to, mint (such as peppermint or spearmint), menthol, cinnamon, vanilla, artificial vanilla, chocolate, artificial chocolate or bubblegum. Natural fruit flavors, artificial fruit flavors or mixtures thereof include, and are not limited to, cherry, grape, orange, strawberry or lemon. Flavor enhancers include, and are not limited to, citric acid. A flavoring agent used in the taste masking composition has a range of from about 0.02 to about 0.06 grams per 100 mL. Preferably, a flavoring agent is present in a range of from about 0.03 to about 0.04 grams per 100 mL.

Optional further sweetening agents include, but are not limited to, sugar sweeteners such as monosaccharides, disaccharides and polysaccharides. Examples of suitable sugar sweeteners include but arc not limited to xylose, ribose, glucose, mannose, galactose, fructose, dextrose, sucrose, maltose, partially hydrolyzed starch (such as maltitol syrup) or corn syrup solids and sugar alcohols such as sorbitol, xylitol, mannitol, glycerin and combination thereof. Preferably, the type of glycerin used is U.S.P. grade. Preferred as a sugar sweetener is high fructose corn syrup. The amount of sugar sweetener used in the taste masking composition will vary depending on the degree of palatability desired for the liquid pharmaceutical composition. Generally the total amount of sugar sweetener used has a range of from 0 to about 90 grains per 100 mL. Preferably, the amount of sugar sweetener used has a range of from about 50 grams to about 90 grams per 100 mL.

Coloring agents also can be incorporated to provide an appealing color to the liquid formulation. Suitable coloring agents include those that avoid chemical incompatibilities with other ingredients. Example coloring agents include dyes, lake dyes or natural coloring.

A liquid formulation may optionally contain pH stabilizers (such as, but not limited to, hydrochloric acid, lactic acid, citric acid, ascorbic acid, potassium phosphate or sodium phosphate), wetting agents (such as, but not limited to, sodium laurel sulfate or docusate sodium), defoaming agents (such as, but not limited to, simethicone), or electrolytes (such as, but not limited to, sodium chloride, potassium chloride or sodium bicarbonate).

An example liquid formulation contains about 0.01 to about 10% w/v of 11-piperazin-1-yldibenzo[b,f][1,4]thiazepine in water or oil vehicle (or combination thereof), about 0 to about 5% w/v buffer, about 0 to about 3% w/v antioxidant, about 0 to about 0.1% w/v chelating agent, about 0 to about 5% w/v preservative, about 0 to about 10% w/v tonicity adjuster, about 0 to about 70% w/v cyclodextrin, about 0 to about 10% w/v surfactant, and about 0 to about 75% w/v cosolvent.

An example liquid formulation suitable for injection contains about 0.05 to about 10% w/v of 11-piperazin-1-yldibenzo[b,f][1,4]thiazepine in water vehicle, about 1 to about 3% W/V buffer, about 0.1 to about 0.5% w/v antioxidant, about 0.01 to about 0.075% w/v chelating agent, about 0.002 to about 2% w/v preservative, about 0.9 to about 5.5% w/v tonicity adjuster, about 1 to about 45% w/v cyclodextrin, about 0.1 to about 5% w/v surfactant, and about 0.5 to about 50% w/v cosolvent.

In some embodiments, 1 mL of the liquid formulation contains about 0.5 to about 10 mg of 11-piperazin-1-yldibenzo[b,f][1,4]thiazepine in water vehicle.

In some embodiments, 1 mL of the liquid formulation contains about 5 to about 20 mg of 11-piperazin-1-yldibenzo[b,f][1,4]thiazepine in an oil vehicle.

An example aqueous-based liquid formulation suitable for injection can contain per mL about 0.2 to about 0.7 mg of the compound of Formula I, about 12.0 to about 19.0 mg of citric acid, about 4.0 to about 6.0 mg of disodium phosphate, about 0.2 to about 0.7 mg of EDTA, about 8 to about 12 mg of benzyl alcohol, and sodium chloride, and optionally other excipients.

An example aqueous-based liquid formulation suitable for injection can contain per mL about 7 to about 12 mg of the compound of Formula I, about 1 to about 4 mg of acetic acid, about 4 to about 7 mg of sodium acetate, and dextrose, and optionally other excipients.

An example aqueous-based liquid formulation suitable for injection can contain per mL about 0.5 to about 2 mg of the compound of Formula I, about 7 to about 11 mg of lactic acid, sodium hydroxide, and sodium chloride at a pH of about 4.5 to about 5.5, and optionally other excipients.

An example aqueous-based liquid formulation suitable for injection can contain per mL about 0.1 to about 1 mg of the compound of Formula I, about 9 to about 13 mg of citric acid, about 18 to about 25 mg of sodium hydroxide, about 2 to about 5 mg HCl, and sodium chloride, and optionally other excipients.

An example aqueous-based liquid formulation suitable for injection can contain per mL about 7 to about 12 mg of the compound of Formula I, about 175 to about 225 mg of hydroxypropyl-β-cyclodextrin, and dextrose, and optionally other excipients.

An example aqueous-based liquid formulation suitable for injection can contain per mL about 7 to about 12 mg of the compound of Formula I, about 175 to about 225 mg of hydroxypropyl-β-cyclodextrin, about 12 to about 18 mg of citric acid, about 4 to about 7 mg of disodium phosphate, and sodium chloride, and optionally other excipients.

An example liquid formulation suitable for injection contains about 0.05 to about 50% w/v of 11-piperazin-1-yldibenzo[b,f][1,4]thiazepine in oil vehicle, about 0.1 to about 1% w/v antioxidant, about 0.1 to about 5% w/v surfactant, and about 1 to about 10% w/v cosolvent.

An example oil-based liquid formulation suitable for injection contains per mL about 11 to about 18 mg of the compound of Formula I and optionally other excipients.

An example oil-based liquid formulation suitable for injection contains per mL about 11 to about 18 mg of the compound of Formula I and about 8 to about 12 mg of Span 80 and optionally other excipients.

An example oil-based liquid formulation suitable for injection contains per mL about 11 to about 18 mg of the compound of Formula I, about 18 to about 23 mg of Polysorbate 20, and about 2 to about 7 mg of α-tocopherol, and optionally other excipients

An example aqueous-based liquid formulation suitable for oral delivery contains about 0.01 to about 10% w/v of the compound of Formula I in lactic acid buffered solution having a pH of about 3.5 to about 4.5, and optionally other excipients.

An example aqueous-based liquid formulation suitable for oral delivery contains about 0.01 to about 10% w/v of the compound of Formula I in lactic acid buffered solution having a pH of about 3.5 to about 4.5, about 8 to about 12% w/v glycerin USP, about 0.01 to about 0.03% w/v of butylparaben NF, and about 0.02 to about 0.04 propylparaben NF, and optionally other excipients.

An example aqueous-based liquid formulation suitable for oral delivery contains about 0.01 to about 10% w/v of the compound of Formula I in lactic acid buffered solution having a pH of about 3.5 to about 4.5, about 8 to about 12% w/v glycerin USP, about 0.01 to about 0.03% w/v of butylparaben NF, and about 0.02 to about 0.04 propylparaben NF, and optionally other excipients.

An example aqueous-based liquid formulation suitable for oral delivery contains about 0.01 to about 10% w/v of the compound of Formula I in lactic acid buffered solution having a pH of about 3.5 to about 4.5, about 8 to about 12% w/v glycerin USP, about 0.01 to about 0.03% w/v of butylparaben NF, and about 0.02 to about 0.04% w/v propylparaben NF, about 10 to about 20% w/v of sucrose, about 30 to about 50% w/v of high fructose corn syrup, about 10 to about 20% w/v sorbitol solution, about 0.5 to about 1.5% w/v sucralose, and about 0.01 to about 1% w/v flavoring (e.g., peppermint), and optionally other excipients.

Liquid formulations can be prepared in the form of an oral suspension. Accordingly, an oral suspension formulation can contain at least one suspending agent, and optionally one or more flocculating agents, wetting agents, sweeteners, flavorings, or colorants.

Suitable suspending agents include, for example, pre-gelatinized starch, powdered cellulose, microcrystalline cellulose, methylcellulose, ethylmethylcellulose, ethylcellulose, sodium carboxymethylcellulose, hydroxyethylcellulose, hydroxypropylmethylcellulose, ethylhydroxyethylcellulose, hydroxypropylcellulose, attapulgite (colloidal magnesium aluminum silicate), bentonite (colloidal aluminum silicate), hectorite (colloidal magnesium aluminum silicate), sepiolite (magnesium silicate), magnesium aluminum silicate, silica gel, colloidal silicon dioxide, acacia, agar, carrageenan, guar gum, karaya gum, locust bean gum, pectin, sodium alginate, propylene glycol alginate, tamarind gum, tragacanth, xanthan gum, carbomer, povidone, polyethylene glycols, gelatin, glycyrrhizin and sodium starch glycolate.

Suitable wetting agents include, for example, surfactants, hydrophilic polymers, clays, glycerin, propylene glycol and ethanol.

The oral liquid suspension can further include stabilizer such as a suitable surfactant, hydrophilic polymer, natural gum, clay, buffer or electrolyte.

Liquid oral suspension formulations of the invention can contain the active ingredient in an amount within the range of about 0.01 to about 20% weight by volume of the final suspension, for example about 0.5 to about 15%, such as about 1 to about 10%, and particularly about 1 to about 5% weight by volume of the final suspension.

Typically one or more suspending agents will be present in the liquid suspension in an amount about 0.1% to about 10% weight by volume of the final suspension.

Typically one or more wetting agents will be present in the liquid suspension in an amount about 0.01% to about 10% weight by volume of the final suspension.

Typically one or more stabilizing agents will be present in the liquid suspension in an amount of about 0.01% to about 10% weight by volume of the final suspension.

Typically one or more preserving agents will be present in the suspension in an amount of about 0.01% to about 10% weight by volume of the final suspension.

An example aqueous-based oral suspension liquid formulation contains about 1 to about 3% w/v of the compound of Formula I, about 5 to about 15% w/v glycerin, about 25 to about 35% w/v sucrose, about 1 to about 5% w/v xantham gum, about 0.01 to about 0.1% w/v Polysorbate 80, about 0.1 to about 0.5% w/v sodium benzoate, about 0.1 to about 0.7% w/v artificial flavoring, and optionally other ingredients.

Other suitable buffers, antioxidants, chelating agents, preservatives, tonicity adjusters, cyclodextrins, surfactants, suspending agents, wetting agents, stabilizers, flocculating agents, sweeteners, flavorings, colorants, cosolvents, preserving agents, taste masking systems and other excipients which may be used are described in Handbook of Pharmaceutical Recipients, 2^{nd} Edition, American Pharmaceutical Association; The Theory and Practice of Industrial Pharmacy/Third Edition, Lachman, Leon, et ill, 1986; Pharmaceutical Dosage Forms: Disperse Systems Volume 1, 2, Lieberman, Herbert A., et al, 1938; Modern Pharmaceutics, Banker, Gilbert and Rhodes, Christopher T, 4th Edition, 2002; and Remington: The Science and Practice of Pharmacy, 20^{th} Edition, 2000, each of which is incorporated herein by reference in its entirety.

The formulations of the invention can include, in addition to 11-piperazin-1-yldibenzo[b,f][1,4]thiazepine, a further active ingredient. Example further active ingredients include benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, serotonin reuptake inhibitors or a mood stabilizer.

Exemplary benzodiazepines include but are not limited to adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, triazolam and equivalents thereof.

Exemplary 5-HT_{1A} and/or 5HT_{1B} ligands include but are not limited to buspirone, alnespirone, elzasonan, ipsapirone, gepirone, zopiclone and equivalents thereof.

Exemplary mGluR2 agonists may include (1S,3R)-1-aminocyclopentane-1,3-dicarboxylic acid, (2S,3S,4S)alpha-(carboxycyclopropyl)glycine, and 3,5-dihydroxyphenylglycine.

Exemplary antidepressants include but are not limited to maprotiline, amitriptyline, clomipramine, desipramine, doxepin, imipramine, nortryptyline, protriptyline, trimipramine, SSRIs and SNRIs such as fluoxetine, paroxetine, citalopram, escitalopram, sertraline, venlafaxine, fluoxamine, and reboxetine.

Exemplary antipsychotics include but are not limited to clozapine, risperidone, quetiapine, olanzapine, amisulpride, sulpiride, zotepine, chlorpromazine, haloperidol, ziprasidone, and sertindole.

Exemplary mood stabilizers may include but are not limited to Valproic acid (valproate) and its derivative (e.g. divalproex), lamotrigine, lithium, verapamil, carbamazepine and gabapentin.

The formulations of the invention can be obtained by conventional procedures using conventional techniques. For example, the active ingredient is typically mixed with an excipient, diluted by an excipient or enclosed within such a carrier in the form of, for example, a capsule or other container. Thus, the compositions can be in the form of elixirs, suspensions, emulsions, solutions, syrups, aerosols, ointments containing, for example, up to 10% by weight of the active compound, liquid-filled gelatin capsules, sterile injectable solutions, and the like.

The formulation of the invention can be administered by any route including orally, intramuscularly, topically, intranasally, intraperitoneally, intrathoracially, intravenously, epidurally, intrathecally, intracerebroventricularly and by injection into the joints.

The amount of active ingredient that is combined with one or more excipients to produce a single dosage form will necessarily vary depending upon the host treated and the particular route of administration. The size of the dose for therapeutic or prophylactic purposes of the active compound(s) will naturally vary according to the nature and severity of the symptoms or conditions, the age and sex of the animal or patient and the route of administration, according to well known principles of medicine.

The present invention further provides methods of treating at least one symptom or condition associated with schizophrenia and other psychotic disorders (e.g., psychotic disorder, psychosis); dementia and other cognitive disorders, anxiety disorders (e.g., generalized anxiety disorder); mood disorders (e.g., depressive disorders, major depressive disorders; bipolar disorders including bipolar I and II, bipolar mania, bipolar depression); sleep disorders; disorders usually first diagnosed in infancy, childhood, or adolescence (e.g., attention-deficit disorder and disruptive behavior disorders); and neurodegenerative disorders comprising administering to a mammal a pharmaceutically effective amount of a liquid formulation of the invention or composition containing one or more of the same. In some embodiments, the symptoms and conditions include but are not limited to anxiety, agitation, hostility, panic, eating disorders, affective symptoms, mood symptoms, negative and positive psychotic symptoms commonly associated with psychosis and neurodegenerative disorders. In some embodiments, the symptoms and conditions are any of psychosis, schizophrenia, bipolar I, and anxiety.

In some embodiments, the present invention further provides methods of treating at least one symptom or condition associated with but not limited to: 1) Schizophrenia and other Psychotic Disorders including but not limited to Psychotic Disorder, Schizophreniform Disorder, Schizoaffective Disorder, Delusional Disorder, Brief Psychotic Disorder, Shared Psychotic Disorder, and Psychotic Disorder Due to a General Medical Condition; 2) Dementia and other Cognitive Disorders; 3) Anxiety Disorders including but not limited to Panic Disorder Without Agoraphobia, Panic Disorder With Agoraphobia, Agoraphobia Without History of Panic Disorder, Specific Phobia, Social Phobia, Obsessive-Compulsive Disorder, Postraumatic Stress Disorder, Acute Stress Disorder, Generalized Anxiety Disorder and Generalized Anxiety Disorder Due to a General Medical Condition; 4) Mood Disorders including but not limited to a) Depressive Disorders, including but not limited to Major Depressive Disorder and Dysthymic Disorder and b) Bipolar Depression and/or Bipolar mania including but not limited to Bipolar I Disorder, including but not limited to those with manic, depressive or mixed episodes, and Bipolar II Disorder, c) Cyclothymic Disorder, d) Mood Disorder Due to a General Medical Condition; 5) Sleep Disorders; 6) Disorders Usually First Diagnosed in Infancy, Childhood, or Adolescence including but not limited to Mental Retardation, Learning Disorders, Motor Skills Disorder, Communication Disorders, Pervasive Developmental Disorders, Attention-Deficit and Disruptive Behavior Disorders, Feeding and Eating Disorders of Infancy or Early Childhood, Tic Disorders, and Elimination Disorders; 7) Substance-Related Disorders including but not limited to Substance Dependence, Substance Abuse, Substance Intoxication, Substance Withdrawal, Alcohol-Related Disorders, Amphetamine (or Amphetamine-Like)-Related Disorders, Caffeine-Related Disorders, Cannabis-Related Disorders, Cocaine-Related Disorders, Hallucinogen-Related Disorders, Inhalant-Related Disorders, Nicotine-Related Disorders, Opioid-Related Disorders, Phencyclidine (or Phencyclidine-Like)-Related Disorders, and Sedative-, Hypnotic- or Anxiolytic-Related Disorders; 8) Attention-Deficit and Disruptive Behavior Disorders; 9) Eating Disorders; 10) Personality Disorders including but not limited to Obsessive-Compulsive Personality Disorder; and 11) Impulse-Control Disorders, by administering to a patient (e.g., a mammal) a pharmaceutically effective amount of a liquid formulation described herein.

The above conditions and disorders are defined for example in the American Psychiatric Association Diagnostic and Statistical Manual of Mental Disorders, Fourth Edition, Text Revision, Washington, D.C., American Psychiatric Association, 2000. Substance abuse and substance dependence and related disorders are also defined therein. This Manual may also be referred to for greater detail on the symptoms and diagnostic features associated with substance use, abuse and dependence. Typical substances that lead to substance abuse and substance dependence include drugs such as amphetamines, cannabis, cocaine, crack, hallucinogenic agents, inhalants, opioids, phencyclidine, sedatives, hypnotics, anxiolytic agents and alcohol. Nicotine can also lead to substance dependence.

In some embodiments, the symptoms and conditions that may be treated using an effective amount of a liquid formulation of the invention include Depressive Disorders (e.g., Major Depressive Disorder), Anxiety Disorders (e.g., Generalized Anxiety Disorder), and Substance-Related Disorders.

The present invention further provides methods of treating at least one symptom or condition described herein by administering to a mammal a pharmaceutically effective amount of a liquid formulation of the invention and a therapeutically effective amount of at least one other therapeutically active agent selected from benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, serotonin reuptake inhibitors, and mood stabilizers.

Administration of two or more active agents can be carried out in combination, e.g., as part of the same formulation, or separately (e.g., serially or consecutively) as part of an appropriate dose regimen designed to obtain the benefits of combination therapy. The appropriate dose regimen, the amount of each dose of an active agent administered, and the specific intervals between doses of each active agent will depend upon the subject being treated, the specific active agent being administered and the nature and severity of the specific disorder or condition being treated.

In general, the formulations provided herein can be administered to a mammal in an amount up to about 750 mg of active agent per day, particularly from about 75 mg to about 750 mg per day, in single or divided doses. In another aspect of the invention, the formulations provided herein may be administered to a mammal in an amount from about 1 mg to about 600 mg per day. In a further aspect of the invention, the formulations provided herein may be administered in an amount from about 100 mg to about 400 mg per day. The formulation may be administered on a regimen of up to 6 times per day, or 1 to 4 times per day. Variations can occur depending upon the mammal being treated and the individual response to the treatment, as well as on the type of pharmaceutical formulation chosen and the time period and interval at which such administration is carried out. In some instances, dosage levels below the lower limit of the aforesaid range may be more than adequate, while in other cases larger doses may be employed to achieve the desired effect, provided that such larger doses are first divided into several small doses for administration throughout the day.

In some embodiments, the formulation is administered comprising a predetermined dosage to a mammal between one and four times a day, wherein the predetermined dosage is from about 1 mg to about 600 mg.

The present invention also provides a method of treating the symptoms or conditions provided herein comprising the step of administering an initial predetermined dosage of the active ingredient in a formulation of the invention to a human patient twice a day, wherein the predetermined dosage is between 1 mg and 30 mg with increases in increments of 1-50 mg twice daily on the second and third day as tolerated. Thereafter, further dosage adjustments can be made at intervals of 2 days or greater.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A clinician may determine the effective amount by using numerous methods already known in the art. The term “treating” within the context of the present invention encompasses to administer an effective amount of the compound of Formula I, or its pharmaceutically acceptable salts, to mitigate either a pre-existing disease state, acute or chronic, or a recurring symptom or condition. This definition also encompasses prophylactic therapies for prevention of recurring conditions and continued therapy for chronic disorders.

The term “mammal” is meant to refer to any warm-blooded animal, preferably a human. In some embodiments, the mammal is in need of treatment because it is suffering from or prone to developing one or more of the symptoms, diseases or disorders described above.

Any or all of the liquid formulations described herein, including any combination thereof, can be used in the preparation of a medicament for the treatment of any of the diseases, disorders, or conditions described herein.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of 11-piperazin-1-yldibenzo[b,f][1,4]thiazepine

### Preparation A (Form A)

Aqueous solution (584 mL; e.g., prepared by extraction of 11-piperazin-1-yldibenzo[b,f][1,4]thiazepine into water/HCl from a toluene solution such as described below in Preparation B) containing 11-piperazin-1-yldibenzo[b,f][1,4]thiazepine hydrochloride was charged to a jacketed 1 L flask. The flask was then charged with toluene (500 mL) and sodium hydroxide (48% w/w, 33.0 g). The mixture was stirred at 70° C. for 30 minutes and became white and cloudy. The mixture was then allowed to settle for 30 min and the phases were separated. The toluene layer was washed at 70° C. with 2×100 mL of water (1^{st} wash=pH 10.3; 2^{nd} wash=pH 8.0). The final toluene volume was 560 mL containing about 74 g of 11-piperazin-1-yldibenzo[b,f][1,4]thiazepine in good purity.

The above procedure was repeated for an additional four aqueous solutions of 11-piperazin-1-yldibenzo[b,f][1,4]thiazepine hydrochloride and the five resulting toluene solutions were combined and evaporated to dryness on a rotary evaporator. The resulting hard solid was then charged to a jacketed vessel and slurried with methyl-t-butyl ether (MTBE) (500 mL). The resulting slurry was stirred overnight at ambient temperature and then cooled to 5° C. and held for 4 h. The solid 11-piperazin-1-yldibenzo[b,f][1,4]thiazepine product was isolated on a no. 3 sinter and washed with 200 mL of cold MTBE. The cake was dried in a vacuum oven overnight at 60° C. yielding 373 g of product.

### Preparation B (Form A)

A toluene solution of 11-piperazin-1-yldibenzo[b,f][1,4]thiazepine (1500 mL, 0.686 mol) prepared by reaction of piperazine with 11-chloro-dibenzo[b,f][1,4]-thiazepine in toluene (see, e.g., U.S. Pat. No. 4,879,288) was treated with 1500 mL deionized water and 90 mL of HCl (32% w/w). The resulting mixture was heated to 70° C. and agitated for 45 min. Agitation was ceased and the mixture allowed to settle and phase separate for 30 min. The lower aqueous phase, containing the HCl salt of 11-piperazin-1-yldibenzo[b,f][1,4]thiazepine was isolated. The aqueous phase was then treated with 1000 mL of toluene and 99 g of aqueous NaOH (47% w/w). The resulting mixture was heated to 70° C. and agitated for 45 min. Agitation was ceased and the mixture allowed to settle and phase separate for 30 min. The lower aqueous phase was discarded and the upper organic phase retained to which 300 mL of deionized water was added. The resulting mixture was agitated for 15 min and then allowed to settle for 30 min. The aqueous phase was discarded and the organic phase retained. The organic phase was extracted once more with 300 mL of deionized water. About 750 mL of toluene from the organic phase was distilled out. The resulting concentrate was cooled to 60° C., then 200 mL of methyl-t-butyl ether (MTBE) was added. The resulting mixture was cooled to ambient temperature then seeded with Form A seed crystals. The seeded mixture was then cooled to 10° C. and held at this temperature for 3 hours under slow agitation. The resulting solid was isolated under suction via a no. 3 sinter. The solid product was then washed with 120 mL of MTBE at ambient temperature and dried at 40° C. under vacuum resulting in 175 g (86.4%) of crystalline product. Assay 99.9% w/w by HPLC area %.

Solid 11-piperazin-1-yldibenzo[b,f][1,4]thiazepine (30 g, 0.1016 mol) prepared as described above was slurried in isopropanol (120 mL). The resulting mixture was warmed to about 63-64° C. to completely dissolve the solid. The resulting solution was filtered through a preheated (about 55° C.) split Buchner funnel fitted with filter paper with a pore size of 6 μm. The filtered solution was then adjusted to 55° C. and seeded with seed crystals of Form A (0.024 g). The seeded solution was maintained at 55° C. for about 2 h then linearly cooled to 40° C. over the course of 6 h, linearly cooled to 20° C. over the course of 2 h, and then linearly cooled to 0° C. over the course of 1 h. The resulting slurry was held at 0° C. for 12 h and the solid product cake (13 mm high×68 mm diameter) was isolated by filtration. The product cake was displacement washed with 30 mL isopropanol prechilled to 0° C. and the cake allowed to deliquor. The product was then dried at 40° C. under vacuum yielding 24.9 g (83%) of Form A. Assay by NMR: 98.9% w/w.

X-ray powder diffraction peak data of crystalline Form A is provided below in Chart A.

CHART A
(Form A)

| Angle | Intensity | |
|---|---|---|
| 2-Theta° | Count | Intensity % |
| 10.8 | 18321 | 51.4 |
| 12.3 | 2390 | 6.7 |
| 13.3 | 24555 | 68.9 |
| 15.2 | 12193 | 34.2 |
| 15.3 | 9799 | 27.5 |
| 16.0 | 2414 | 6.8 |
| 17.2 | 18803 | 52.7 |
| 18.8 | 6502 | 18.2 |
| 19.3 | 7290 | 20.4 |
| 20.0 | 3666 | 10.3 |
| 20.4 | 15535 | 43.6 |
| 21.2 | 25874 | 72.6 |
| 21.7 | 16902 | 47.4 |
| 22.1 | 1473 | 4.1 |
| 24.1 | 3968 | 11.1 |
| 24.2 | 2197 | 6.2 |
| 24.9 | 3579 | 10 |
| 25.5 | 35663 | 100 |
| 26.4 | 6298 | 17.7 |
| 27.9 | 3290 | 9.2 |
| 28.0 | 3746 | 10.5 |
| 28.3 | 2206 | 6.2 |
| 28.6 | 2711 | 7.6 |
| 28.9 | 2142 | 6 |
| 29.4 | 4006 | 11.2 |
| 29.8 | 2464 | 6.9 |
| 30.4 | 2754 | 7.7 |
| 30.9 | 5213 | 14.6 |
| 31.0 | 5143 | 14.4 |
| 31.6 | 2053 | 5.8 |
| 32.1 | 3643 | 10.2 |
| 32.4 | 4234 | 11.9 |
| 32.5 | 3827 | 10.7 |
| 33.2 | 2102 | 5.9 |
| 34.6 | 1540 | 4.3 |
| 35.8 | 1543 | 4.3 |
| 36.3 | 3768 | 10.6 |
| 36.9 | 3086 | 8.7 |
| 38.1 | 2062 | 5.8 |
| 39.0 | 2801 | 7.9 |
| 39.4 | 1492 | 4.2 |

### Preparation C (Amorphous)

Into a 1000 mL round-bottom flask equipped with a magnetic stirring bar and reflux condenser with a nitrogen inlet was charged with 25.0 g (0.110 mol) of dibenzo[b,f][1,4]thiazepine-11(10-H)-one (made by the method of J. Schmutz et al. Helv. Chim. Acta., 48: 336 (1965)), as a dry solid, followed by 310 mL POCl₃ and 3 mL of N,N-dimethylaniline. The reaction mixture was heated at reflux (106° C.) for 6 hours giving a clear orange solution. The reaction was then cooled to room temperature, and POCl₃ removed on the rotary evaporator leaving an orange oil. This residue was partitioned between ice-water (500 mL) and ethyl acetate (800 mL). The layers were separated and the aqueous phase extracted with ethyl acetate (3×200 mL). The combined ethyl acetate extracts were dried over MgSO₄, filtered, and then stripped down on the rotary evaporator, leaving the crude imino chloride as a light yellow solid (26.26 g, 97% yield). The structure was confirmed by NMR and mass spectroscopy (300 MHz, CDCl₃; ES+, M+1=246.7). Crude imino chloride (27.35 g, 0.111 mol) was added to 1000 mL o-xylene in a 2000 mL round-bottom flask equipped with a magnetic stir bar and a reflux condenser with nitrogen inlet. To this solution was added commercially available piperazine (47.95 g, 0.557 mol) in one portion as a dry solid at room temperature. The mixture was stirred until nearly all the piperazine dissolved. Then the reaction mixture was heated at reflux (142° C.) for 40 hours (out of convenience). The reaction was then allowed to cool to room temperature, and an aliquot was partitioned between 1 N NaOH/CH₂Cl₂. The organic phase was checked by TLC (silica gel, CH₂Cl₂/methanol 90:10, iodoplatinate visualized) and showed clean conversion to one major product (Rf=0.45). A drop of the reaction solution was diluted with CH₃CN to prepare a sample for LC/MS analysis, which confirmed the presence of the desired product (M+1=296.4). The reaction mixture was stripped down on the rotary evaporator under high vacuum to remove the xylene. The residue was partitioned between 1 N NaOH (400 mL) and CH₂Cl₂ (200 mL). The layers were separated, and the aqueous phase further extracted with CH₂Cl₂ (3×200 mL). The combined CH₂Cl₂ extracts were washed with brine (200 mL), then dried over MgSO₄, filtered, and stripped down on the rotary evaporator to give the crude title compound as a yellow gum (35.3 g). The crude free base was purified by flash column chromatography over silica gel (600 g) eluting with a gradient of 0 to 20% methanol in CH₂Cl₂. Fractions containing the pure desired product were combined and stripped down on the rotary evaporator, to afford the purified free base as a light yellow foam (25.67 g, 78% yield).

### Example 2

### Stability of Form A

Individual samples of Form A were slurried in various solvents (acetone, ethanol, ethyl acetate, methylethyl ketone, toluene, and water). The mixtures were stirred overnight at room temperature in sealed containers. The samples were then filtered and vacuum dried at 50° C. for 2 h. The resulting material in each of the solvents tested was a white crystalline material having an XRPD diffraction pattern consistent with Form A. Accordingly, Form A has good stability in a variety of solvents and workup conditions.

### Example 3

### Thermal Analysis of Form A

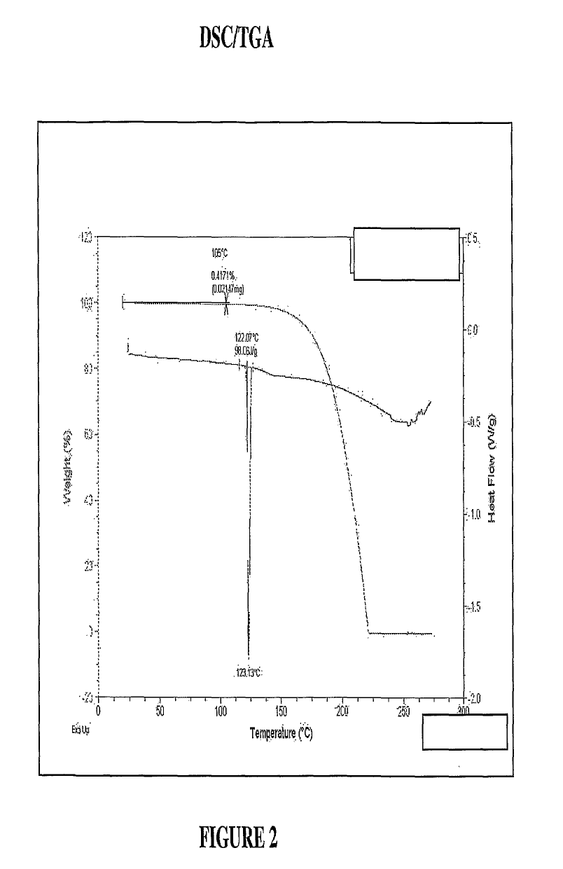

DSC and TGA data consistent with Form A are provided in FIG. 2. The DSC data displayed one sharp endothermic event at 123.1° C. which corresponded to a melt event prior to degradation. The TGA data shows 0.4% weight loss in the water/solvent region.

### Example 4

### Dynamic Vapor Sorption Analysis of Form A

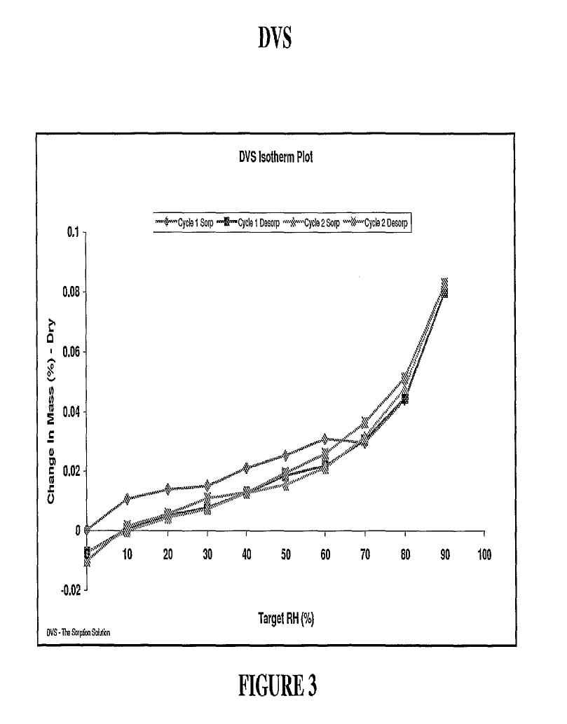

DVS data of Form A revealed that the crystalline form is non-hygroscopic showing only slight, reversible water gain without hysteresis. As shown in FIG. 3, two cycles overlay well with no evidence of form change.

### Example 5

### Injection Formulations

Example formulations for injection are provided below. Stock solutions of acid and/or base salts are prepared in water for injection. 11-piperazin-1-yldibenzo[b,f][1,4]thiazepine is dissolved in the acid solution or in the prepared buffer. Additional components such as antioxidant(s), preservative(s), chelating agent(s), and tonicity modifiers are added to the solubilized 11-piperazin-1-yldibenzo[b,f][1,4]thiazepine as needed for stability, package compatibility and in vivo compatibility. The solution is made to final volume with water or buffer for injection. The solution can be sterilized by filtration through a 0.22 μm filter. Percentages are w/v.

TABLE P

| | |
|---|---|
| Compound of Formula I | 0.05-10% |
| Buffer | 1-3% |
| Antioxidant | 0.1-0.5% |
| Chelating agent | 0.01-0.075% |
| Preservative | 0.002-2% |
| Tonicity adjustment | 0.9-5.5% |
| Cyclodextrin | 1-45% |
| Surfactant | 0.1-5% |
| Cosolvent | 0.5%-50% |

| | |
|---|---|
| Immediate release parenteral solution | Per mL water |

P1

| | | |
|---|---|---|
| Formula I | 0.5 | mg |
| Citric Acid | 15.5 | mg |
| Disodium Phosphate | 5.6 | mg |
| Ethylenediamine tetrancetic acid (salt) | 0.5 | mg |
| Benzyl Alcohol | 10 | mg |
| Sodium chloride | qs to 300 | mOsm/kg |
| Water for Injection | qs ad 1 | ml |

P2

| | | |
|---|---|---|
| Formula I | 9 | mg |
| Acetic Acid | 2 | mg |
| Sodium Acetate | 5.5 | mg |
| Dextrose | qs to 300 | mOsm/kg |
| Water for Injection | qs ad 1 | mL |

P3

| Formula I | 1 | mg |
|---|---|---|
| Lactic Acid | 9 | mg |
| Sodium Hydroxide | qs to pH 5 | |
| Sodium Chloride | qs to 300 | mOsm/kg |
| Water for Injection | qs ad 1 | mL |

P4

| | | |
|---|---|---|
| Formula I | 0.5 | mg |
| Citric Acid | 11.6 | mg |
| Sodium Hydroxide | 22 | mg |
| Hydrochloric Acid | 3.65 | mg |
| Sodium Chloride | qs to 300 | mOsm/kg |
| Water for Injection | qs ad 1 | mL |

An additional formulation for injection containing a cyclodextrin is provided below in Table Q. A stock solution of the cyclodextrin is prepared in water for injection. The compound of Formula I is stabilized in the cyclodextrin solution. A tonicity modifying agent such as dextrose is added if the solution is hypoosmotic. The solution is made to final volume with water or cyclodextrin solution.

TABLE Q

| | | |
|---|---|---|
| Formula I | 10 | mg |
| Hydroxypropyl-β-cyclodextrin | 200 | mg |
| Dextrose | qs to 300 | mOsm/kg |
| Water for Injection | qs ad 1 | mL |

An additional formulation for injection containing a cyclodextrin in a buffered solution is provided below in Table R. The compound of Formula I is solubilized in the cyclodextrin solution which is acidified or buffered. A tonicity modifying agent such as sodium chloride is added if the solution is hypoosmotic. The solution is made to final volume with water or cyclodextrin solution.

TABLE R

| | | |
|---|---|---|
| Formula I | 10 | mg |
| Hydroxypropyl-β-cyclodextrin | 200 | mg |
| Citric Acid | 15.5 | mg |
| Disodium Phosphate | 5.6 | mg |
| Sodium Chloride | qs to 300 | mOsm/kg |
| Water for Injection | qs ad 1 | mL |

Liquid formulations in oil suitable for injection are provided in Table S below. The excipients are solubilized in the oil. The compound of Formula I is dissolved in the oil vehicle. The mixture is made to final volume with oil. The formulation or its components are sterilized by irradiation.

TABLE S

| | |
|---|---|
| Formula I | 0.05-50% |
| Oil | 20-99% |
| Antioxidant | 0.1-1% |
| Cosolvent | 1-10% |
| Surfactant | 0.1-5% |

S1

| | | |
|---|---|---|
| Formula I | 15 | mg |
| Polysorbate 20 | 20 | mg |
| α-tocopherol | 5 | mg |
| Soybean Oil | qs ad 1 | mL |

### Example 6

### Oral Solution Formulations

Examples of non-taste masked solution include:

EXAMPLE 6a
Formula I Liquid Solution (0.1 mg/mL)

| Component | % W/V |
|---|---|
| Formula I | 0.01 |
| Lactic Acid/Sodium Lactate pH 4 Buffer, 0.3M | (qs to 100% w/v) |

### Manufacturing Procedure for Example 13a

Using the appropriate equipment and conditions for the desired batch size, a solution is prepared as follows:
1. Add and dissolve the compound of Formula I in 90% of the final volume of buffer using an appropriate mixer.
2. Add buffer as needed to bring the batch to the final batch weight
3. Mix for an appropriate amount of time, thereby forming the solution.

EXAMPLE 6b
Formula I Liquid Solution (60 mg/mL)

| Component | % W/V |
|---|---|
| Formula I | 6 |
| Lactic Acid, 0.3M Solution | (qs to 100% w/v) |

### Manufacturing Procedure

Using the appropriate equipment and conditions for the desired batch size, a solution is prepared as follows:
1. Add and dissolve the compound of Formula I in 90% of the final volume of 0.3M lactic acid solution using an appropriate mixer.
2. Add 0.3 M lactic acid solution as needed to bring the batch to the final batch weight.
3. Mix for an appropriate amount of time, thereby forming the solution.

### Example 6c

| Component | % W/V |
|---|---|
| Formula I | 0.01 |
| Glycerin, USP | 10 |
| Butylparaben, NF | 0.02 |
| Propylparaben, NF | 0.03 |
| Lactic Acid/Sodium Lactate pH 4 Buffer, 0.3M | (qs to 100% w/v) |

### Manufacturing Procedure

Using the appropriate equipment and conditions for the desired batch size, a solution is prepared as follows:
1. Add und dissolve the compound of Formula I in 70% of the final volume of buffer solution in a first container using an appropriate mixer;
2. Prepare a paraben-glycerin slurry by placing the glycerin in a second container, adding butylparaben and propylparaben and dispersing the parabens with an appropriate mixer;
3. Transfer the paraben-glycerin slurry to the first container and mix with an appropriate mixer for an appropriate amount of time;
4. Add buffer solution as needed to bring the batch to the final batch weight;
5. Mix for an appropriate amount of time, thereby forming the solution.

### Example 6d

| Component | % W/V |
|---|---|
| Formula I | 6 |
| Glycerin, USP | 10 |
| Butylparaben, NF | 0.02 |
| Propylparaben, NF | 0.03 |
| Lactic Acid Solution 0.3M | (qs to 100% w/v) |

### Manufacturing Procedure

Using the appropriate equipment and conditions for the desired batch size, a solution is prepared as follows:
1. Add and dissolve the compound of Formula I in 70% of the final volume of 0.3M lactic acid solution in a first container using an appropriate mixer;
2. Prepare a paraben-glycerin slurry by placing the glycerin in a second container, adding butylparaben and propylparaben and dispersing the parabens with an appropriate mixer;
3. Transfer the paraben-glycerin slurry to the first container and mix with an appropriate mixer for an appropriate amount of time;
4. Add 0.3 M lactic acid solution as needed to bring the batch to the final batch weight;
5. Mix for an appropriate amount of time, thereby forming the solution.

Examples of taste-masked solutions may include the following formulations.

### Example 6e

| Component | % W/V |
|---|---|
| Formula I | 6 |
| Lactic Acid Solution | As needed |
| Glycerin, USP | 10 |
| Purified Water | 5 |
| Sucrose, Extra Fine Granular NF | 15 |
| High Fructose Corn Syrup | 40 |
| Sorbitol Solution, USP | 15 |
| Butylparaben, NF | 0.02 |
| Propylparaben, NF | 0.03 |
| Sucralose, NF | 1.02 |
| Peppermint Flavor | 0.04 |
| Purified Water | (qs to 100% w/v) |

### Manufacturing Procedure

Using the appropriate equipment and conditions for the desired batch size, a taste masked pharmaceutical solution is prepared as follows:
1. Add a sorbitol solution and an appropriate amount of purified water in a first container;
2. Mix the sorbitol solution and water with an appropriate mixer;
3. Prepare a paraben-glycerin slurry by placing the glycerin in a second container, adding butylparaben and propylparaben and dispersing the parabens with an appropriate mixer;
4. Transfer the paraben-glycerin slurry to the first container and mix with an appropriate mixer for an appropriate amount of time;
5. Add the remaining liquid ingredients to the first container and mix with an appropriate mixer for tin appropriate amount of time;
6. Add the dry ingredients and the pharmaceutically active agent to the first container and mix with tin appropriate mixer for an appropriate amount of time;
7. Add the flavoring agents to the first container and mix with an appropriate mixer;
8. Adjust pH to a desired value as needed by adding an appropriate acid or base;
9. Add purified water as needed to bring the batch to the final batch weight; and,
10. Mix the ingredients, slurry and agents for an appropriate amount of time, thereby forming the solution.

### Example 7

### Liquid Suspensions

An example oral liquid suspension formulation is provided below in Table T.

TABLE T

| Ingredients | Unit Amount (gram %) |
|---|---|
| Formula I | 2.0 |
| Glycerin | 10.0 |
| Sucrose | 30 |
| Xanthan Gum | 2 |
| Polysorbate 80 | 0.05 |
| Sodium Benzoate | 0.20 |
| Artificial Flavoring | 0.45 |
| Purified Water, USP | qs to 100.00 ml |

The liquid oral suspension formulation can be made as follows:
1. Dry blend 20% of the final weight of sucrose with the xanthan gum in a suitable blender for approximately 10 minutes;
2. Mix 50% of the final volume of water and the glycerin in a suitable vessel for approximately 5 minutes;
3. Add the dry blend from step 1 to the water and mix until dispersed, approximately 10 minutes;
4. Add and dissolve the remaining sucrose, mixing for approximately 10 minutes;
5. Add polysorbate 80 and sodium benzoate and mix approximately 10 minutes;
6. Add the compound of Formula I (screened through a 40 mesh screen) and mix approximately 10 minutes;
7. Add the flavoring and mix approximately 10 minutes;
8. Bring suspension to near final volume and mix for approximately 10 minutes;
9. Let suspension deaerate for approximately 12-16 hours;
10. Bring suspension to final volume with water and mix for approximately 10 minutes.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A liquid formulation comprising about 0.005 to about 60% w/v of 11-piperazin-1-yldibenzo[b,f][1,4]thiazepine in a liquid vehicle.

2. The liquid formulation of claim 1 further comprising at least one pharmaceutical excipient selected from a buffer, an antioxidant, a chelating agent, a preservative, a tonicity adjuster, a cyclodextrin, a surfactant, a suspending agent, a wetting agent, a stabilizer, a flocculating agent, a sweetener, a flavoring, a colorant, and a cosolvent.

3. The liquid formulation of claim 1 comprising about 0.005 to about 25% w/v 11-piperazin-1-yldibenzo[b,f][1,4]thiazepine.

4. The liquid formulation of claim 1 comprising about 0.01 to about 10% w/v of 11-piperazin-1-yldibenzo[b,f][1,4]thiazepine.

5. The liquid formulation of claim 1 comprising about 0 to about 5% w/v buffer.

6. The liquid formulation of claim 1 comprising about 0 to about 3% w/v weight antioxidant.

7. The liquid formulation of claim 1 comprising about 0 to about 0.1% w/v chelating agent.

8. The liquid formulation of claim 1 comprising about 0 to about 5% w/v preservative.

9. The liquid formulation of claim 1 comprising about 0 to about 10% w/v tonicity adjuster.

10. The liquid formulation of claim 1 comprising about 0 to about 70% w/v cyclodextrin.

11. The liquid formulation of claim 1 comprising about 0 to about 10% w/v surfactant.

12. The liquid formulation of claim 1 comprising about 0 to about 75% w/v cosolvent.

13. The liquid formulation of claim 1 comprising about 0.01 to about 10% w/v of 11-piperazin-1-yldibenzo[b,f][1,4]thiazepine, about 0 to about 5% w/v buffer, about 0 to about 3% w/v antioxidant, about 0 to about 0.1% w/v chelating agent, about 0 to about 5% w/v preservative, about 0 to about 10% w/v tonicity adjuster, about 0 to about 70% w/v cyclodextrin, about 0 to about 10% w/v surfactant, and about 0 to about 75% w/v cosolvent.

14. The liquid formulation of claim 1 wherein 1 mL of said formulation comprises about 0.5 to about 10 mg of 11-piperazin-1-yldibenzo[b,f][1,4]thiazepine in water vehicle.

15. The liquid formulation of claim 1 wherein 1 mL of said formulation comprises about 5 to about 20 mg of 11-piperazin-1-yldibenzo[b,f][1,4]thiazepine in an oil vehicle.

16. The liquid formulation of claim 1 wherein said liquid vehicle comprises an aqueous buffered solution having a pH of about 3 to about 6.

17. The liquid formulation of claim 16 wherein said pH is about 4 to about 4.5.

18. The liquid formulation of claim 1 further comprising a sweetener or flavoring agent.

19. The liquid formulation of claim 1 which is suitable for injection.

20. The liquid formulation of claim 1 which is suitable for oral administration.
